(19) United States
(12) Patent Application Publication
(10) Pub. No.: US 2026/0016407 A1
(43) Pub. Date: Jan. 15, 2026

(54) Method for Determining the Quantity of at Least One Pulverulent Binder in a Mixture Comprising Wood Particles

(71) Applicant: Flooring Technologies Ltd., Kalkara (MT)

(72) Inventors: Norbert Kalwa, Horn-Bad Meinberg (DE); Jinming Zhang, Berlin (DE)

(21) Appl. No.: 18/880,919

(22) PCT Filed: Jun. 22, 2023
(86) PCT No.: PCT/EP2023/066999

§ 371 Date: Jan. 3, 2025

(30) Foreign Application Priority Data
Jul. 4, 2022 (EP) 22182896.5

(51) Int. Cl. G01N 21/359; G01N 21/31; G01N 21/3563; G01N 33/46

(52) U.S. Cl. CPC G01N 21/359; G01N 21/3563; G01N 33/46; G01N 2021/3196

(57) ABSTRACT

A method for determining the amount of at least one biodegradable powdery binder in a mixture with wood particles. The method comprises the steps of: providing mixtures of at least one powdery binder and wood particles as reference samples, wherein the at least one powdery binder and wood particles are present in the mixtures in quantitatively defined mixing ratios in each case, recording of at least one NIR spectrum of the reference samples, providing a mixture to be measured consisting of at least one powdery binder and wood particles, recording of at least one NIR spectrum of the mixture of the at least one powdery binder with wood particles, and determining the quantitative amount of powdery binder in the mixture of powdery binder and wood particles by comparison with the NIR spectra recorded for the reference samples.

### CROSS-REFERENCE TO RELATED APPLICATIONS

This application is the United States national phase of International Patent Application No. PCT/EP2023/066999 filed Jun. 22, 2023, and claims priority to European Patent Application No. 22182896.5 filed Jul. 4, 2022, the disclosures of each of which are hereby incorporated by reference in their entireties.

### BACKGROUND

### Technical Field

The disclosure relates to a method for determining or measuring the amount of at least one powdery binder in a mixture with wood particles and a method for producing wood-based panels from this mixture of powdery binder and wood particles using this measuring method.

### Technical Considerations

Wood-based panels, such as chipboard or wood fiberboard, whereby wood fiberboard always refers to medium or high-density wood fiberboard (MDF/HDF), form the basis of many everyday objects, such as furniture or coverings for walls, floors or ceilings. OSB boards (oriented strand boards) are used in timber and prefabricated house construction, as OSB boards are lightweight and still meet the structural requirements placed on building boards. OSB boards are used as building boards and as wall or roof cladding or also in flooring.

The wood-based panels listed are manufactured in multi-stage processes, whereby the first step in each of these processes is the mixing of the corresponding wood particles with a suitable binder, followed by spreading the mixture onto a conveyor belt and pressing the spread mixture into a wood particle mat or panel. The binders or glues typically used are usually based on urea-formaldehyde, phenol-formaldehyde or PMDI (polymeric diphenylmethane diisocyanate) glues.

The wood-based materials industry is also increasingly being called upon to replace these petroleum-based components with renewable raw materials. There is currently a wide range of products to choose from when using glues based on renewable raw materials. To avoid competition between use as food and use as glue, more and more residues from the production of food arising anyways are being.

Residual materials from other industrial production processes are also used. These come, for example, from the production of cellulose (lignin) or from crop waste (rape seed residue, etc.). However, products that are used as food are also used (sugar, starch, soy flour). Lignin is a macromolecule based on phenolic components. Soy flour and rape seed residue contain proteins and oils as their main components. Sugars and starches are carbohydrates.

These glue alternatives are produced in powder form and, with the exception of sugar, are not readily soluble in water. If the existing technical equipment is to be used, they can only be used as dispersions in production.

However, the petroleum-based glues described above are all liquids that can be homogeneously dosed onto the wood particles or fibers using spray systems in mixers or gluing drums. This dosing method has been used in production for decades and has been further optimized in terms of the distribution of the glue and the required quantity, e.g., through nozzle optimization or high-pressure gluing.

Dispersions are rather unfavorable for glues based on renewable raw materials, as dispersions introduce a relatively large amount of water into the system, which is problematic for the process. Too much water in the chip or fiber cake can lead to steam splitting, which should be avoided by post-drying the chips/fibers.

For these reasons, dosing as a powder is a more suitable alternative. This is done via dosing screws, which apply the powder to the wood particles/fibers. Further distribution can then take place in mixers, for example.

However, the distribution on the wood matrix can vary greatly depending on the powder. It is unclear whether the existing mixers even enable homogeneous distribution. The distribution of the glue particles on the wood particles can at best be assessed visually. However, the particle sizes of the powders range from 1 to 200 μm, which makes this assessment difficult. Some of the powders also have a similar color to the wood particles, which also makes an objective assessment of the powder distribution difficult. Furthermore, neither a continuous nor an in-line assessment is possible during production. This means that in order to achieve certain minimum values for strength and swelling, a higher dosage of glue powder must be used. This leads to unnecessary raw material consumption and higher costs.

One possible solution to this problem would be to color the powder or use a UV-active substance, which would then allow the distribution to be assessed with the help of a special lamp. However, this would first require a homogeneous distribution of the colorant or UV-active compound in the powder and would generate additional costs. In addition, it is only possible to assess whether a change in dosage results in better distribution by determining the technological values on the product. This can lead to faulty production/rejects.

There are disadvantages to the manufacturing processes used to date. For example, the production lines for manufacturing wood-based panels are optimized for liquid glues. If powdery glues or binders are used, the lack of analytics can lead to an unnecessary increase in dosage, which is associated with higher costs. In addition, unclear production circumstances can occur.

### SUMMARY

The present disclosure is based on the technical object of providing an analysis method that can be used to continuously determine the powder distribution on the wood particles during production. This should be possible at any point in the production process and provide a large amount of data quickly thanks to a high measurement frequency. The measurements should be non-destructive and not require a great deal of technical effort. It should not be necessary to make changes to the systems to install the measurement analytics. The conditions in production at the various possible measuring points should not interfere with the measurements.

This object is solved by a method having features as described herein.

### BRIEF DESCRIPTION OF THE DRAWINGS

The disclosure is explained in more detail below with reference to the figures.

FIG. 1 shows NIR spectra of wood chips, soy flour and mixtures of soy flour and wood chips;

FIG. 2 shows NIR spectra of wood chips, starch and mixtures of starch and wood chips; and

FIG. 3 shows a section of NIR spectra of wood chips and mixtures of starch and wood chips.

### DETAILED DESCRIPTION

In some non-limiting embodiments, a method for determining the amount of at least one biodegradable powdery binder in a mixture with wood particles is provided, the method comprising the following steps:
Providing mixtures of at least one powdery binder and wood particles as reference samples, wherein the at least one powdery binder and wood particles are present in the mixtures in quantitatively defined mixing ratios,
Recording of at least one near-infrared radiation (NIR) spectrum of the reference samples using at least one NIR measuring head in a wavelength range of 900 nm to 1700 nm (nanometers),
Providing a mixture to be measured consisting of at least one powdery binder and wood particles,
Recording at least one NIR spectrum of the mixture of the at least one powdery binder and wood particles using the at least one NIR measuring head in a wavelength range of 900 nm to 1700 nm, and
Determining the quantitative proportion of powdery binder in the mixture of powdery binder and wood particles by comparison with the NIR spectra recorded for the reference samples.

According to the present disclosure, NIR spectroscopy is used to determine the quantity of a powdery binder and, if applicable, the composition of a combination of two or more powdery binders in a mixture with wood particles, whereby the method can be used in the ongoing production operation of wood-based panels. The NIR measuring method can be carried out at various positions or process steps in the production of wood-based panels, as will be explained in detail below. For example, the NIR measuring head, for example the NIR multi-measuring head, can not only take into account a single measuring position, but can also be guided in a traversing manner over a chip, strand or fiber cake. This makes it possible, for example, to detect edge effects that frequently occur during the production of wood-based materials. It is also advantageous to measure individual layers (top layer/middle layer). A further advantage is that other parameters (e.g.: moisture, see for example EP 2 915 658 B1, EP 2 808636 B1) can also be analyzed from the NIR spectra. It is known that glues based on renewable raw materials require certain minimum amounts of moisture for curing. In addition, the moisture also improves the adhesion of the glue to the wood particles or fibers. This can also help to avoid quality defects.

The present method makes it possible to provide the measured values in a short time (online, preferably without disruptive time delay) compared to conventional (known) measurement methods. The measurement data can be used for quality assurance, research and development, process control, process regulation, process control, etc. The measurement process does not reduce the production speed, etc. Basically, it improves the monitoring of production. In addition, downtimes are also reduced through quality determinations and system adjustments.

The determination of the amount of powdery binder or binder mixture possible with the present method is preferably carried out exclusively by means of NIR measurement. A combination with other spectroscopic methods, using other wavelengths outside the NIR range, is not intended.

An NIR measuring head is therefore used, preferably an NIR multi-measuring head, which allows the amount of powdery binder to be determined by recording spectral data (spectra) in the near-infrared range (700-2000 nm). The NIR radiation interacts with the organic functional groups, such as O—H, C—H and N—H, which are present in the binders. During the interaction, the NIR radiation is scattered and reflected by the measured sample. By receiving the reflected NIR radiation via an NIR detector, an NIR spectrum is generated. During this measurement, a large number of individual NIR measurements are carried out in one second, so that statistical validation of the values is also guaranteed. NIR spectroscopy offers the possibility of establishing a direct link between the spectral information (NIR spectra) and the parameters of the binders to be determined.

The present method takes advantage of the fact that the NIR radiation penetrates to a certain extent into the near-surface areas of the material, but the majority of the NIR radiation is reflected or scattered at the surface of a wood particle mixture or a wood particle cake. The reflected or scattered NIR radiation is detected by the NIR detector and the NIR spectrum determined is used to determine the desired parameters (in this case the quantity of powdery binder applied).

Spectral data from the entire recorded spectral range is preferably used to determine the amount of powdery binder, i.e., a whole range of several wavelengths is used rather than a single, discrete wavelength.

According to the method according to the present disclosure, mixtures of at least one powdery binder and wood particles are thus initially provided as reference samples, the at least one powdery binder and the wood particles being present in the mixtures in quantitatively defined mixing ratios in each case. To provide the reference samples, various quantities of powdery binder are mixed with wood particles, e.g., 5% by weight, 7% by weight, 10% by weight, 15% by weight of powdery binder relative to the total quantity of the binder-wood particle mixture.

It should also be noted that the reference sample is identical to the sample to be measured; i.e., the binder-wood particle mixture of the reference sample has the same composition as the binder-wood particle mixture to be measured. The similarity of the sample to be measured and the reference sample may be important when using additives such as flame retardants, fibers and/or other additives.

At least one NIR spectrum is recorded from these reference samples in a wavelength range of 900 nm to 1700 nm, preferably 1400 nm to 1700 nm, more preferably 1450 nm to 1650 nm, or 1500 nm to 1600 nm.

The different quantitative quantities of powdery binders of the reference samples are then assigned to the respective NIR spectra of these reference samples, and a correlation is established between the spectral data of the NIR spectra of the reference samples and the corresponding binder quantities as parameter values, i.e., an NIR spectrum of the reference sample corresponds to each parameter value of the reference sample.

Subsequently, at least one mixture of at least one powdery binder and wood particles is provided, and at least one NIR spectrum of the mixture of the at least one powdery binder with wood particles is recorded using the at least one NIR measuring head in a wavelength range of 700 nm to 2000 nm. The quantitative amount or proportion of the powdery binder in the mixture of powdery binder and wood particles can then be determined by comparison with the NIR spectra recorded for the reference samples.

As already mentioned, a comparison and interpretation of the NIR spectra is sensibly carried out over the entire recorded spectral range. Thus, in some non-limiting embodiments, spectral data from the NIR spectral range of 900 nm to 1700 nm are used to determine the amount of at least one powdery binder in a mixture with wood particles. In some non-limiting embodiments, spectral data from the NIR spectral range of 1400 nm to 1700 nm, preferably 1450 nm to 1650 nm, more preferably 1500 nm to 1600 nm, are used to determine the amount of at least one powdery binder in a mixture with wood particles. In some non-limiting embodiments, spectral data from the NIR spectral ranging from 900 nm to 1100 nm, preferably 900 nm to 1000 nm, are used to determine the amount of at least one powdery binder in a mixture with wood particles.

In some non-limiting embodiments of the present disclosure, the amount of the powdery binder in the mixture with the wood particles is 5 to 50% by weight, preferably 7 to 40% by weight, more preferably 10 to 30% by weight, or 15 to 20% by weight. In some non-limiting embodiments, the amount of the powdery binder in the mixture with the wood particles is 5% by weight, 7% by weight, 10% by weight or 15% by weight (based on the total amount of the binder-wood particle mixture).

The measuring methods of the present disclosure can be used to determine the amount of any type of powdery binder, wherein biodegradable binders are preferred.

Biodegradable binders can be either naturally occurring binders or synthetic binders.

If a natural binder is used as a biodegradable binder, it is selected from a group comprising starch, cellulose derivatives, such as carboxymethyl cellulose, chitosan, and/or gluten-containing binders, such as hide glue, bone glue, and/or leather glue; binders containing milk proteins, for example from the group of caseins, and binders containing plant proteins, for example from the group of soy binders, agar-agar, alginate, gelatine, guar gum, gum arabic, xanthan gum, pectins, and/or locust bean gum, and/or polysaccharides such as carrageenan, lignin and/or rapeseed pomace. The use of starch and soy flour is preferred.

In some non-limiting embodiments of the present wood fiber mat, the starch used as a binder is selected from the group comprising potato starch, corn starch, wheat starch, and/or rice starch.

Starch is a polysaccharide with the formula (C₆H₁₀O₅)ₙ which consists of α-D-glucose units. The macromolecule is therefore a carbohydrate. Starch can physically bind, swell and gelatinize many times its own weight in water when exposed to heat. When heated with water, the starch swells at 47-57° C., the layers burst, and at 55-87° C. (potato starch at 62.5° C., wheat starch at 67.5° C.) starch paste is formed, which has varying degrees of stiffness depending on the type of starch (corn starch paste has greater stiffness than wheat starch paste, the latter has greater stiffness than potato starch paste) and decomposes more or less easily with acidification.

Starch can be used as a binder both in its native form and in modified (derivatized) form. Thus, the at least one starch can be present in native and/or modified (derivatized) form in the wood fibre mat. Preferably, DuraBinders from Ecosynthetix is used as the starch-containing binder.

If modified or derivatized starch is used as a binder, it can be selected from a group comprising cationic starch, anionic starch, carboxylated starch, carboxy-methylated starch, sulfated starch, phosphorylated starch, etherified starch such as hydroxyalkylated starch (e.g., hydroxyethylated starch, and/or hydroxypropylated starch), oxidized starch containing carboxyl or dialdehyde groups and/or hydrophobic starches such as acetate, succinate, and/or half or phosphate esters.

It is also generally conceivable to use a mixture of a natural starch and a derivatized starch or of several natural starches and/or several derivatized starches.

The particle size of the starch is 20 to 100 μm, preferably 30 to 80 μm, more preferably 40 to 60 μm, e.g., 50 μm.

In some non-limiting embodiments of the present disclosure, spectral data from the NIR spectral ranging from 900 nm to 1100 nm, preferably 900 nm to 1000 nm, are used to determine the amount of starch in a mixture with wood particles when using starch as a powdery binder.

The particle size of the soy flour used is 30 to 300 μm, preferably 50 to 200 μm, more preferably 60 to 100 μm, e.g., 70 μm.

In some non-limiting embodiments of the present disclosure, spectral data from the NIR spectral range of 1450 nm to 1650 nm, preferably 1500 nm to 1600 nm, are used to determine the amount of soy flour in a mixture with wood particles when using soy flour as a powdery binder.

The present spectroscopic method also makes it possible to determine the quantity and composition of a combination of at least two powdery binders in a mixture with wood particles. This results from the fact that different spectral ranges of the NIR spectrum can be used for evaluation.

In such a combination, for example, a first powdery binder and a second powdery binder may be present in a ratio of 10% by weight: 90% by weight to 90% by weight: 10% by weight, preferably 25% by weight: 75% by weight to 75% by weight: 25% by weight, more preferably 55% by weight: 45% by weight to 45% by weight: 55% by weight, for example 50: 50% by weight. A preferred combination may consist, for example, of starch and soy flour.

When using binder mixtures, it may be necessary to create a calibration model using multivariate data analysis (MDA) for the reference samples. In multivariate analysis methods, several statistical variables are typically analyzed simultaneously in a known manner. For this purpose, these methods usually reduce the number of variables contained in a data set without simultaneously reducing the information contained therein. In the present case, the multivariate data analysis is carried out using the partial least squares regression (PLS) method, which allows a suitable calibration model to be created. The data obtained is preferably evaluated using suitable analysis software, such as the SIMCA-P analysis software from Umetrics AB or The Unscrambler from CAMO.

If a synthetic binder is used as a biodegradable binder, this is preferably selected from the group comprising saponified polyvinyl alcohol, polycaprolactam-polyamide, polylactate, aliphatic polyester resins, for example polybutylene succinate, polybutylene succinate-adipate, polyethylene-polypropylene composite resin, polylactates being preferred. In a preferred embodiment, polylactic acid fibers with a length of 38 mm+/−3 mm and a fineness of 1.7 dtex are used.

As already indicated above, wood fibers, wood chips and/or wood strands are preferably used as wood particles.

Wood fibers or chips can be obtained by chipping the wood chips in a chipper or a refining process of the wood chips in a refiner.

The wood fibers typically used to manufacture the wood fiber boards, for example dry wood fibers, have a length of 1.5 mm to 20 mm and a thickness of 0.05 mm to 1 mm.

The wood strands used to produce OSB can have a length of 50 to 200 mm, preferably 70 to 180 mm, more preferably 90 to 150 mm; a width of 5 to 50 mm, preferably 10 to 30 mm, more preferably 15 to 20 mm; and a thickness of 0.1 to 2 mm, preferably between 0, 3 and 1.5 mm, more preferably between 0, 4 and 1 mm.

However, it is also generally conceivable to use the present measuring method for other primary products derived from renewable raw materials for the production of panels or molded parts (e.g., straw, hemp, and/or bagasse, etc.).

As already indicated above, the method according to the present disclosure for determining the amount of powdery binder in a wood particle mixture can be carried out continuously and online in a production line for manufacturing wood-based panels. In some non-limiting embodiments, the method can be carried out in an automatically controlled system with an alarm message. In some non-limiting embodiments, however, the determination of the amount of at least one powdery binder in a mixture with wood particles can also be carried out offline.

The amount of powdery binder in a wood particle mixture can be determined at various process stations in the production line for manufacturing wood-based panels.

A method of manufacturing wood-based panels using a powdery binder comprises the following steps:
a) Production of wood particles from suitable woods,
b) Interim storage of the wood particles, if necessary, especially in silos or bunkers,
c) Drying the wood particles,
d) Sorting or sifting the wood particles according to the size of the wood particles,
e) Mixing the wood particles with at least one powdery binder, e.g., using dosing screws;
f) Applying the mixture of wood particles and the at least one powdery binder to a conveyor belt by means of air and/or throw sifting, and
g) Pressing the mixture of wood particles and the at least one powdery binder arranged on the conveyor belt.

The determination of the amount of the at least one powdery binder in the mixture with wood particles using the measuring method according to the present disclosure can be carried out, for example
i) after mixing the wood particles with the at least one powdery binder but before applying the mixture of wood particles and the at least one powdery binder to a conveyor belt (i.e., after step e), and/or
ii) after the mixture of wood particles and the at least one powdery binder has been applied to a conveyor belt by means of air and/or throw sifting, but before the mixture of wood particles and the at least one powdery binder arranged on the conveyor belt has been pressed (i.e., after step f), and/or
iii) after pressing the mixture of wood particles and the at least one powdery binder arranged on the conveyor belt to form a wood particle cake (i.e., after step g).

It can also be advantageous to measure individual layers, such as the top layer/middle layer in the production of chipboard and OSB. In addition, NIR not only measures the surface, but also penetrates into areas close to the surface. This is particularly useful for analyzing the effects of scattering (wind/throw scattering).

In some non-limiting embodiments, the measurement of the binder-wood particle mixture deposited on a conveyor belt and the wood particle cake (i.e., chip, strand or fiber cake) obtained after pressing can be carried out by traversing. The at least one NIR measuring head moves across the entire width of the deposited or pressed binder-wood particle mixture in the production line, transverse to the direction of travel of the deposited or pressed binder-wood particle mixture, in order to analyze certain problem areas, for example shortfalls in the edge or middle area of the deposited or pressed binder-wood particle mixture. This makes it possible, for example, to detect edge effects that frequently occur during the production of wood-based panels.

A method is thus provided in which, by using an NIR measuring head, the quantity of a powdery binder in a binder-wood particle mixture can be determined from a single NIR spectrum or the reflection or scattering of NIR radiation, by means of a non-contact measurement. In some non-limiting embodiments of the present disclosure, the data determined with the measuring head or measuring heads are used directly for system control or regulation.

In addition, in some non-limiting embodiments of the present disclosure, the storage of the data makes it possible to improve quality control. The stored data can also make an advantageous contribution to the evaluation of system tests, e.g., commissioning of a system during new installation or after maintenance or repair or for in-situ testing of new production or measurement processes. The immediate availability of the measured values and the high measurement frequency enable very close monitoring, control and/or regulation of the systems.

The advantages of this method are manifold: non-contact multi-parameter determination (“real time” or “real-time” measurement) with significantly reduced time delay in the evaluation of the measured parameter values; improved system control and/or regulation, reduction of rejects, improvement of the quality of the products manufactured on the system, and/or improvement of system availability.

The control system of the respective production plant comprises at least one computer-aided evaluation unit (or processor unit) and a database. In the evaluation unit, the NIR spectrum measured for the product (i.e., coated substrate material) is compared with the calibration models created for the individual parameters. The parameter data determined in this way is stored in the database.

The data determined using this spectroscopic method can be used to control the respective production line. The non-contact measured parameter values of the NIR multi-measurement head (“actual values”) can, as described above, be used directly and in “real time” for the control or regulation of the relevant system, for example by storing the actual values measured and stored in the database, e.g., a relational database, and comparing them with the target values of these parameters available there. The resulting differences are then used to control or regulate the production line.

A computer-implemented method and a computer program product comprising instructions which, when the program is executed by at least one processor, cause the processor to execute the computer-implemented method, are provided for the adjustment and control of the respective production line. The computer program is stored in a memory unit of the control system of the respective production line. In some non-limiting embodiments, the at least one processor may be implemented in hardware, firmware, or a combination of hardware and software.

The following describes in detail methods for manufacturing wood fiberboards, chipboards and OSB in which the measuring method according to the present disclosure can be used.

Fiberboard and particleboard are usually manufactured in a process involving the following steps:
a) Production of wood chips from suitable wood,
b) Chipping the wood chips into wood shavings or wood fibers,
c) Temporary storage of wood chips or wood fibers, e.g., in silos or bunkers,
d) Drying the wood chips or wood fibers,
e) Sorting or sifting the wood chips or wood fibers according to the size of the wood chips or wood fibers,
f) If necessary, further shredding of the wood chips or wood fibers and intermediate storage,
g) Mixing the wood chips or wood fibers with at least one powdery binding agent, e.g., using dosing screws;
h) applying the mixture of wood chips or wood fibers and the at least one powdery binder to a conveyor belt by means of air and/or throw sifting, and
i) Pressing the wood chips or wood fibers arranged on the conveyor belt.

The determination of the amount of the at least one powdery binder in the mixture with wood chips/wood fibers using the measuring method according to the present disclosure can be carried out
i) after mixing the wood chips/wood fibers with the at least one powdery binder but before applying the mixture of wood chips/wood fibers and the at least one powdery binder to a conveyor belt (i.e., after step g), and/or
ii) after the mixture of wood chips/wood fibers and the at least one powdery binder has been applied to a conveyor belt by means of air and/or throw sifting, but before the mixture of wood chips/wood fibers and the at least one powdery binder arranged on the conveyor belt has been pressed (i.e., after step h), and/or
iii) after pressing the mixture of wood chips/wood fibers and the at least one powdery binder arranged on the conveyor belt into a wood particle cake (i.e., after step i).

The processes for manufacturing particleboard and wood fiberboard differ with regard to the size and composition of the wood fibers or wood chips used and with regard to the pressures and temperatures used. However, the essential process sequence and thus the sequence of the process steps are similar for all boards and are known to the person skilled in the art.

In the case of wood fiber boards, the mixture of wood fibers and binder is spread on a conveyor belt to form a single-layer fiber cake, which is subjected to prepressing before hot pressing. Accordingly, an (additional) NIR measurement in accordance with the method according to the present disclosure would also be conceivable between the prepressing and hot pressing steps.

In the case of chipboard, the mixture of wood chips and binder is spread on a conveyor belt to form a multi-layer chip cake, with the wood chips being spread on top of each other as a first surface layer, middle layer and second surface layer. Accordingly, an (additional) NIR measurement according to the method of the present disclosure would be conceivable in each case after the individual layers have been scattered, i.e., after the first top layer/after the middle layer/after the second top layer.

OSB boards are also manufactured in a multi-stage process, whereby the strands of debarked round wood, preferably softwood, are first peeled lengthwise using rotating knives. In the subsequent drying process, the natural moisture of the strands is reduced at high temperatures. The moisture content of the strands can vary depending on the binder used, although the moisture content should be well below 10% to avoid splitting during subsequent pressing. Depending on the binder, wetting may be more favorable on moist strands or on dry strands. In addition, as little moisture as possible should be present in the strands during the pressing process in order to reduce the vapor pressure generated during the pressing process as much as possible, as this could otherwise cause the raw board to burst.

Once the strands have dried, they are mixed with a suitable binder. The mixture of strands and binder is then spread alternately lengthwise and crosswise to the production direction in spreading equipment, so that the strands are arranged crosswise in at least three layers (lower surface layer-middle layer-upper surface layer). The spreading direction of the lower and upper surface layer is the same, but differs from the spreading direction of the middle layer. The strands used in the top layer and middle layer also differ from each other. For example, the strands used in the top layers are flat and the strands used in the middle layer are less flat or even chip-shaped. Usually, two material strands are used in the production of OSB boards: one with flat strands for the subsequent surface layers and one with “chips” for the middle layer. Accordingly, the strands in the middle layer can be of poorer quality, as the bending strength is essentially generated by the surface layers. For this reason, fines produced during machining can also be used in the middle layer of OSB boards.

Once the strands have been scattered, they are continuously pressed under high pressure and at high temperatures of 200 to 250° C., for example.

Accordingly, an NIR measurement according to the measuring method according to the present disclosure would be possible in each case after mixing the strands with the at least one powdery binder, after spreading the individual layers, i.e., after the first top layer/after the middle layer/after the second top layer, and/or after pressing the mixture of wood strands and binder spread on a conveyor belt.

### Example 1: Mixture of Wood Chips and Soy Flour

Various amounts of soy flour (Prolia) were added to surface layer chips for the production of chipboard (5, 7, 10 and 15% by weight). The soy flour had an average particle size of 70 μm (max. particle size <200 μm). The chips and the flour were mixed homogeneously using a laboratory mill. The mixtures were then analyzed on a plate using an NIR measuring head. A chip sample and a soy flour sample were also measured.

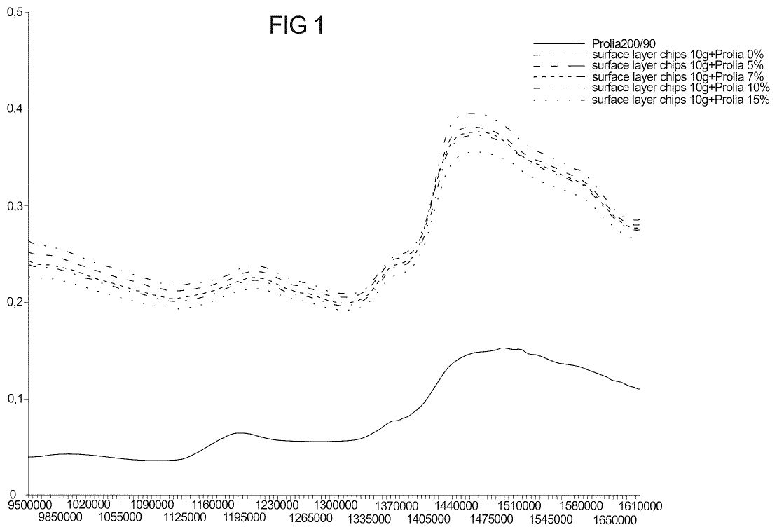

As it turned out, a clear gradation between the individual concentrations was recognizable. Calibration/evaluation is best possible at the peak at approx. 1550-1560 nm (see diagram in FIG. 1).

### Example 2: Mixture of Wood Chips and Starch

Various amounts of corn starch were added to surface layer chips for the production of chipboard (5, 10 and 15 wt %). The corn starch had an average particle size of 50 μm (max. particle size <100 μm). The chips and starch were mixed homogeneously using a laboratory mill. The mixtures were then analyzed on a plate using an NIR measuring head.

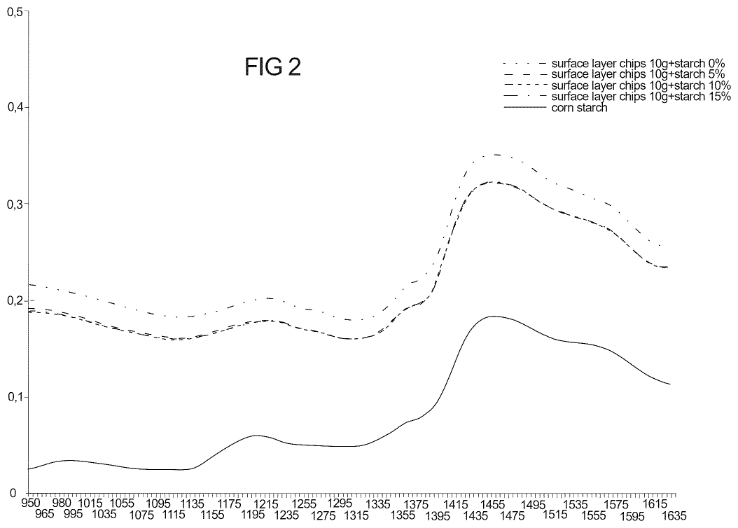

A chip sample and a starch sample were also measured. As it turned out, in the range of 1550 to 1560 nm a clearly weaker gradation between the individual concentrations was recognizable. This is particularly true for 5 and 10 wt % (see diagram in FIG. 2).

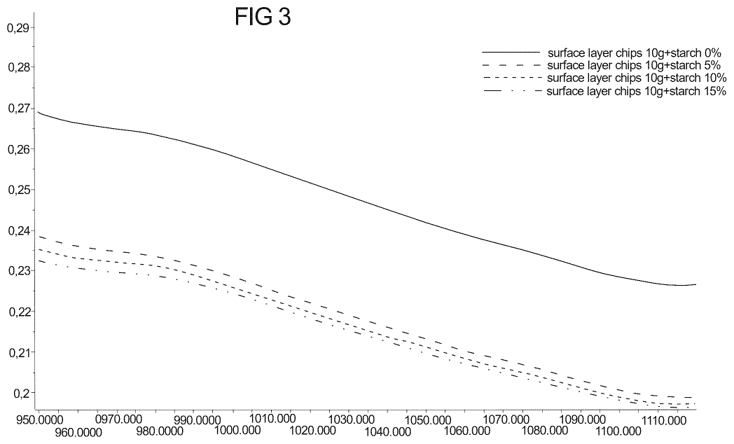

For this reason, the range 950-995 nm is selected for calibration. There, the spectra are separated with increasing starch content (see diagram in FIG. 3).

As it turns out, it is possible to reliably determine different powder glues. The concentrations determined are in the concentration ranges that would also be used in the production of wood based material. A NIR measuring head can therefore be used to determine the concentrations of biopowder glues on particulate or fibrous wood as well as their fluctuations. As the peaks used for evaluation are located in different areas of the NIR spectrum, combinations of different bio-glues can also be analyzed.

1. A method for determining the amount of at least one biodegradable powdery binder in a mixture with wood particles,
comprising the steps of
Providing mixtures of at least one powdery binder and wood particles as reference samples, wherein the at least one powdery binder and wood particles are present in the mixtures in quantitatively defined mixing ratios in each case,
Recording of at least one NIR spectrum of the reference samples using at least one NIR measuring head in a wavelength range of 900 nm to 1700 nm,
Providing a mixture to be measured consisting of at least one powdery binder and wood particles,
Recording of at least one NIR spectrum of the mixture of the at least one powdery binder with wood particles using the at least one NIR measuring head in a wavelength range of 900 nm to 1700 nm, and
Determining the quantitative amount of powdery binder in the mixture of powdery binder and wood particles by comparison with the NIR spectra recorded for the reference samples.

2. The method according to claim 1, wherein spectral data from the entire recorded spectral range are used to determine the amount of at least one powdery binder in a mixture with wood particles.

3. The method according to claim 1, wherein spectral data from the NIR spectral range of 900 nm to 1600 nm are used to determine the amount of at least one powdery binder in a mixture with wood particles.

4. The method according to claim 1, wherein spectral data from the NIR spectral range of 1450 nm to 1650 nm, are used to determine the amount of at least one powdery binder in a mixture with wood particles.

5. The method according to claim 1, wherein spectral data from the NIR spectral range of 900 nm to 1100 nm are used to determine the amount of at least one powdery binder in a mixture with wood particles.

6. The method according to claim 1, wherein the quantitative amount of the powdery binder in the mixture with the wood particles is 5 to 50% by weight (based on the total amount of the binder-wood particle mixture).

7. The method according to claim 1, wherein a naturally occurring binder is used as a biodegradable binder.

8. The method according to claim 1, wherein a synthetic binder is used as a biodegradable binder.

9. The method according to claim 1, wherein the wood particles are at least one of wood chips, wood fibres and/or wood strands.

10. The method according to claim 1, wherein the determination of the amount of at least one powdery binder in a mixture with wood particles is carried out continuously online in a production line for wood-based panels.

11. The method according to claim 1, wherein the determination of the amount of at least one powdery binder in a mixture with wood particles is carried out off-line.

12. A process for the production of wood-based panels comprising:
a) Production of wood particles from suitable woods,
b) Interim storage of the wood particles, if necessary,
c) Drying the wood particles,
d) Sorting or sifting the wood particles according to the size of the wood strands,
e) Mixing the wood particles with at least one biodegradable powdery binder;
f) applying the mixture of wood particles and the at least one powdery binder to a conveyor belt by means of air and/or throw sifting, and
g) Pressing the mixture of wood particles and the at least one powdery binder arranged on the conveyor belt,
wherein the determination of the amount of the at least one powdery binder in the mixture with wood particles is carried out according to a method according to claim 1,
i) after mixing the wood particles with the at least one powdery binder but before applying the mixture of wood particles and the at least one powdery binder to a conveyor belt (after step e), and/or
ii) after the mixture of wood particles and the at least one powdery binder has been applied to a conveyor belt by means of air and/or throw sifting, but before the mixture of wood particles and the at least one powdery binder arranged on the conveyor belt has been pressed (according to step f), and/or
iii) After pressing the mixture of wood particles and the at least one powdery binder arranged on the conveyor belt into a wood particle cake (after step g).

13. The method according to claim 4, wherein spectral data from the NIR spectral range of 1500 nm to 1600 nm are used to determine the amount of at least one powdery binder in a mixture with wood particles.

14. The method according to claim 5, wherein spectral data from the NIR spectral range of 900 nm to 1000 nm are used to determine the amount of at least one powdery binder in a mixture with wood particles.

15. The method according to claim 6, wherein the quantitative amount of the powdery binder in the mixture with the wood particles is 7 to 40% by weight (based on the total amount of the binder-wood particle mixture).

16. The method according to claim 7, wherein the naturally occurring binder is selected from a group comprising starch, cellulose derivatives, chitosan, gluten-containing binders, milk-protein-containing binders and/or plant-protein-containing binders.

17. The method according to claim 8, wherein the synthetic binder is selected from the group comprising saponified polyvinyl alcohol, polycaprolactam-polyamide, polylactate, aliphatic polyester resins, polybutylene succinate-adipate, polyethylene-polypropylene composite resin.
